(19) United States
(12) Patent Application Publication
(10) Pub. No.: US 2025/0130155 A1
(43) Pub. Date: Apr. 24, 2025

(54) NON-DARCY FLOW PARAMETERS FOR EVALUATING CAPROCK INTEGRITY ASSOCIATED WITH GEOLOGICAL CO2 SEQUESTRATION AND STORAGE

(71) Applicant: SAUDI ARABIAN OIL COMPANY, Dhahran (SA)

(72) Inventors: Hui-Hai LIU, Houston, TX (US); Jilin Jay ZHANG, Houston, TX (US)

(73) Assignee: SAUDI ARABIAN OIL COMPANY, Dhahran (SA)

(21) Appl. No.: 18/492,543

(22) Filed: Oct. 23, 2023

(51) Int. Cl. G01N 15/08; E21B 41/00; G01N 33/24

(52) U.S. Cl. CPC G01N 15/0826; E21B 41/0064; G01N 33/24

(57) ABSTRACT

Systems and methods for determining caprock integrity for geological sequestration of CO₂, such as in above saline aquifers. The testing system for performing the method includes a core container in fluid communication with an upstream reservoir and an upstream pump, further in fluid communication with a downstream liquid reservoir and a downstream liquid pump, and further in fluid communication with a downstream gas reservoir and a downstream gas pump. The method includes determining transient hydraulic conductivity and hydraulic gradient of a caprock core sample using the testing system based on non-Darcy flow.

### FIELD OF INVENTION

The present disclosure relates generally to methods and systems for use in the oil and gas industry and, more particularly, to methods and systems for determining non-Darcy flow parameters for evaluation of caprock integrity associated with CO₂ sequestration and storage.

### BACKGROUND

Long-term geological sequestration (geosequestration) and storage of carbon dioxide (CO₂) in subterranean formation reservoirs (e.g., saline aquifers or depleted oil and gas reservoirs) offers the possibility of sustaining access to fossil fuels while reducing gaseous emissions that contribute to global warming and climate change. However, prior to implementation, associated risks of gaseous leakage into the atmosphere must be carefully managed to ensure environmental safety. In formation reservoirs, where CO₂ is injected for sequestration and storage, caprock integrity is a critical consideration, but also a complex geo-mechanical issue. As used herein, the term “caprock,” and grammatical variants thereof, refers to a layer of hard, generally (preferably) less permeable rock overlying and sealing a deposit of a gas (although other fluids can also be sealed thereunder) and saline water.

Evaluation and understanding of flow properties of caprock are critical to the design of virtually any project involving CO₂ sequestration and storage. A number of factors can influence caprock integrity including, but not limited to, the thickness of the caprock, the permeability of the caprock, the heterogeneity of the caprock, the geological activity beneath the caprock, and stress state changes that occur throughout injection life. Caprock integrity directly impacts the ability of CO₂ sequestration and storage safety, capacity, and injectivity. Indeed, caprock integrity is critical for withstanding pressures (e.g., injection pressures) that can lead to fractures or other channels that can cause CO₂ gaseous emissions into the atmosphere and caprock integrity that withstands such pressure further permits greater storage capacity of CO₂ within a formation reservoir.

Caprock integrity is measured by caprock sealing capacity. As used herein, the term “caprock sealing capacity,” and grammatical variants thereof, refers to the capability of a caprock to prevent CO₂ leakage or emission from a formation sequestration and storage reservoir. It is critical, therefore, that flow of CO₂ through caprock is evaluated and understood to accurately determine caprock integrity. CO₂ leakage pathways through caprock may be through area leakage and/or leakage through fractures or faults. These leakage pathways are often created by the displacement of water into a formation sequestration and storage reservoir by injected CO₂. Because many of such formation reservoirs have strong water-solid interactions, particularly in tight formations such as caprock, water flow through caprocks (e.g., due to displacement caused by injected CO₂) does not follow Darcy's law and current analytical methods of evaluating caprock integrity do not consider non-Darcy flow behavior of water in CO₂ sequestration and storage reservoirs. Without being bound by theory, it is believed that non-Darcy flow behavior has not been considered for evaluating caprock integrity due to a lack of an efficient and cost-effective method for determining non-Darcy flow parameters.

In view of the above, it is desirable that an accurate assessment of caprock integrity be available for site selection, characterization, and operational evaluation for CO₂ sequestration and storage.

### SUMMARY OF THE DISCLOSURE

Various details of the present disclosure are hereinafter summarized to provide a basic understanding. This summary is not an exhaustive overview of the disclosure and is intended neither to identify certain elements of the disclosure, nor to delineate the scope thereof. Rather, the primary purpose of this summary is to present some concepts of the disclosure in a simplified form prior to the more detailed description that is presented hereinafter.

In one or more aspects, the present disclosure provides a method including determining a hydraulic gradient and a hydraulic conductivity of a caprock core sample based on non-Darcy flow using a testing system comprising a core container comprising an upstream inlet, a downstream outlet, and a confining pressure pump in fluid communication with the core container, an upstream reservoir in fluid communication with the upstream inlet and comprising a first upstream valve for selectively controlling fluid flow between the upstream reservoir and the caprock core sample located within the core container, an upstream pump in fluid communication with the upstream reservoir and comprising a second upstream valve for selectively controlling fluid flow between the upstream pump and the upstream reservoir, a downstream liquid reservoir in fluid communication with the downstream outlet of the core container and comprising a first downstream valve for selectively controlling fluid flow between the downstream liquid reservoir and the caprock core sample located within the core container, a downstream liquid pump in fluid communication with the downstream liquid reservoir and comprising a second downstream valve for selectively controlling fluid flow between the downstream liquid pump and the downstream liquid reservoir, a downstream gas reservoir in fluid communication with the downstream liquid reservoir and comprising a third downstream valve for selectively controlling gaseous flow between the downstream gas reservoir and the downstream liquid reservoir, and a downstream gas pump in fluid communication with the downstream gas reservoir and comprising a fourth downstream valve for selectively for controlling gaseous flow between the downstream gas pump and the downstream gas reservoir. The determining step may include closing the first upstream valve, the second upstream valve, the first downstream valve, the second downstream valve, the third downstream valve, and the fourth downstream valve, arranging the caprock core sample in the core container, wherein the core container is pressurized using the confining pump to a predetermined confining pressure, and equilibrating the testing system by opening the first upstream valve, the second upstream valve, the first downstream valve, and the second downstream valve to saturate the caprock core sample with water, opening the fourth downstream valve, thereby providing gaseous flow to the downstream gas reservoir until a predetermined equilibrium gas pressure in the downstream gas reservoir is reached, closing the second downstream valve and the fourth downstream valve when the predetermined equilibrium gas pressure is reached, opening the third downstream valve, and thereafter, closing the first upstream valve. The determining step may further include performing a flow test by opening the second upstream valve, thereby pressurizing the upstream reservoir using the upstream pump to a predetermined pressure; then keeping constant the pressure in the upstream reservoir, opening the first upstream valve, thereby flowing water between the upstream liquid reservoir and the downstream liquid reservoir through the caprock core sample, measuring flow rate data between the upstream inlet and the downstream outlet as a function of time, and measuring pressure differential data between the upstream outlet and the downstream outlet as a function of time. The method may further include collecting the flow rate data and the pressure differential data, calculating hydraulic conductivity (K) of the caprock core sample as a function of time where

$$K = \frac{{q}_{0} L}{Δ p} + \frac{{AL}^{2}}{6} \frac{d ( ln Δ p )}{dt} ,$$

and q₀ is the flow rate data at the upstream inlet at time zero (0), L is a length of the caprock core sample, A is a storage factor, and Δp is the pressure differential data, and calculating the hydraulic gradient (i) of the caprock core sample as a function of time where

$$i = \frac{1}{L} ( \frac{Δ p}{ρ g} + Δ z ) ,$$

and ρ is porosity of the caprock core sample, g is gravitational acceleration, and Δz is an elevation difference between the upstream inlet and the downstream outlet.

In another aspect, the present disclosure provides a testing system that includes a core container that includes an upstream inlet in fluid communication with the core container, a downstream outlet in fluid communication with the core container, and a confining pressure pump in fluid communication with a of the core container. The system further include an upstream reservoir in fluid communication with the upstream inlet of the core container and comprising a first upstream valve for selectively controlling fluid flow between the upstream reservoir and the caprock core sample located within the core container, an upstream pump in fluid communication with the upstream reservoir and comprising a second upstream valve for selectively controlling fluid flow between the upstream pump and the upstream reservoir, a downstream liquid reservoir in fluid communication with the downstream outlet of the core container and comprising a first downstream valve for selectively controlling fluid flow between the downstream liquid reservoir and the caprock core sample located within the core container, a downstream liquid pump in fluid communication with the downstream liquid reservoir and comprising a second downstream valve for selectively controlling fluid flow between the downstream liquid pump and the downstream liquid reservoir, a downstream gas reservoir in fluid communication with the downstream liquid reservoir and comprising a third downstream valve for selectively controlling gaseous flow between the downstream gas reservoir and the downstream liquid reservoir, and a fourth downstream valve for selectively for controlling gaseous flow between the downstream gas pump and the downstream gas reservoir.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 provides a schematic drawing of gaseous (e.g., CO₂ or H₂) leakage pathways in a caprock of a saline aquifer.

FIG. 2 is a chart showing non-Darcy's flow velocity through caprocks as a function of hydraulic gradient, according to one or more aspects of the present disclosure.

FIG. 3 shows a generalized diagram of an example testing system, according to one or more aspects of the present disclosure.

FIG. 4 shows a detailed diagram of an example testing system, according to one or more aspects of the present disclosure.

FIG. 5 shows an alternative detailed diagram of an example testing system, according to one or more aspects of the present disclosure.

FIG. 6 is a method flowchart for determining the threshold hydraulic pressure using the testing apparatus, according to one or more aspects of the present disclosure.

### DETAILED DESCRIPTION

The present disclosure relates generally to methods and systems for use in the oil and gas industry, and more particularly, to methods and systems for determining non-Darcy flow parameters for evaluation of caprock integrity associated with CO₂ sequestration and storage.

As discussed above, there is increasing interest in the introduction of CO₂ into subterranean formation reservoirs for sequestration and storage. The present disclosure provides a laboratory testing method and system for evaluating caprock integrity to mitigate CO₂ leakage in formation sequestration and storage reservoirs. More specifically, the present disclosure provides a method and system for evaluating caprock integrity based on non-Darcy parameters to determine the relationship between permeability and hydraulic gradient.

Advantageously, the methods and systems of the present disclosure permit accurate caprock integrity analysis for CO₂ sequestration and storage within subterranean formation reservoirs, such as saline aquifers and depleted oil and gas wells. Saline aquifers are generally characterized by great depths that make them often technically and economically unfeasible for exploitation for surface uses; depleted oil and gas wells have previously been drilled into a subterranean formation and can no longer be used for hydrocarbon recovery. Accordingly, advantageously, these subterranean formation reservoirs are either readily available (e.g., saline aquifers) or afford utilization of the large capital expenditures borne during hydrocarbon drilling.

It is to be understood that while the embodiments of the present disclosure are generally described with reference to sequestration and storage of CO₂ in saline aquifer formation reservoirs, the methods and systems described herein are equally applicable to depleted oil and gas wells.

Embodiments of the present disclosure are described in more detail hereinafter with reference to the accompanying Figures. In the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

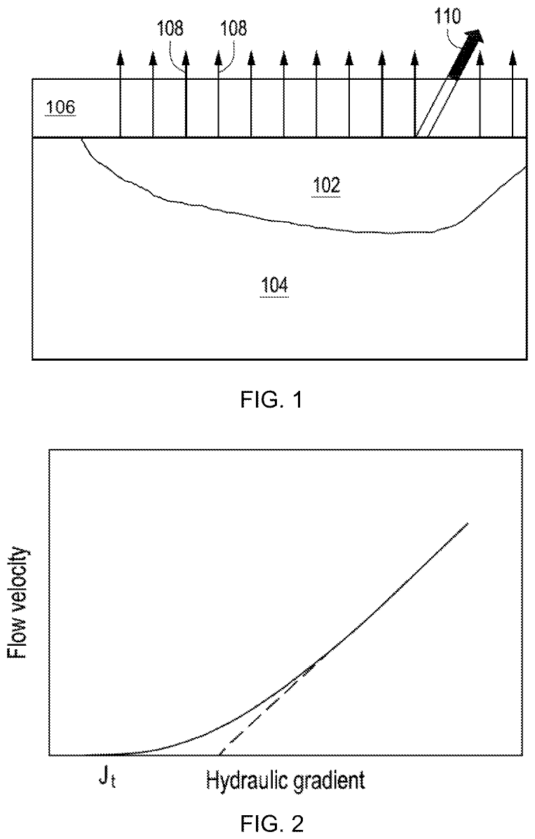

Referring to FIG. 1, illustrated is a schematic drawing of gaseous CO₂ leakage pathways in a caprock of a saline aquifer. As shown, saline aquifer 104 comprises a gaseous CO₂ plume 102 below caprock 106. Various CO₂ leakage pathways may include area leakage 108 (the vertical arrows) or leakage through fractures and faults 110. The area leakage 108 and fracture/fault leakage 110 may provide pathways (generated or reopened) for CO₂ escape through the caprock 106 due to, for example, pore pressure increase owing to the CO₂ plume 102 injected into the saline aquifer 104 below the caprock 106. Area leakage 108 occurs when the stored gaseous plume 102 imbibes into the caprock 106 due to such pore pressure. The existence of fracture/fault leakage 110 occurs due to an existing fracture/fault or due to shifts in the caprock 106 after the CO₂ plume 102 has been injected. Both types of leakage can be formed through injection of the CO₂ plume, causing CO₂ to imbibe the caprock 106 and open avenues that can lead to significant CO₂ escape into the atmosphere.

More specifically, the CO₂ plume 102 is non-wetting, whereas the brine in caprock 106 is wetting (water) and area leakage 108 can occur due to the pore pressure difference between the non-wetting phase and the wetting phase exceeding the break-through pressure of the caprock 106, with the break-through pressure being related to the capillary barrier between the two phases. Both types of leakage have traditionally been considered to occur by assuming that the flow through the caprock 106 follows Darcy's law in which flow velocity through the caprock 106 is linearly proportional to the hydraulic gradient. Darcy's law has thus traditionally been incorporated in evaluation of caprock integrity.

However, flow through tight formations, such as caprock, does not follow Darcy's law due to strong water-solid interactions. Referring now to FIG. 2, illustrated is a chart showing characteristics of non-Darcy's flow through caprocks. As shown, the flow does not occur until the hydraulic gradient exceeds the threshold hydraulic gradient, labeled J, (solid line) in FIG. 2. In such instances, sequestered and stored CO₂ gases below the caprock may not imbibe through the caprock, even after the pore pressure exceeds the breakthrough pressure. Thus, to evaluate the threshold gradient and associated non-Darcy's flow behavior of water through caprock can allow for a more accurate assessment of caprock integrity for geological sequestration and storage of CO₂.

Available caprock integrity measurement techniques to determine the relationship between permeability and hydraulic gradient involve steady-state flow methods in which a single permeability value from caprock tested by running water therethrough one test at a time for a given hydraulic gradient. Essentially, the available traditional methods evaluate caprock integrity using steady-state flow permeability as a function of hydraulic gradient with multiple test runs. Accordingly, traditional caprock integrity evaluation methods are time-consuming and not suitable for routine use in practical applications. Differently, the methodology described herein is based on transient water flow and requires only a single test run, thus making it efficient for practical use.

The methods and testing systems disclosed herein allow for in-lab measurement of the hydraulic conductivity of a caprock core sample as a function of hydraulic gradient with a single test run. The results from the single in-lab test may be used to determine flow characteristics of the caprock from which the caprock core sample was taken. The single test run may be performed for various time periods, as described below.

The methods and systems of the present disclosure to evaluate and ensure caprock integrity analysis can contribute to a reduction in greenhouse gas emissions. CO₂ is a heat-trapping gas that is known to contribute to climate change, warming the climate that can lead to changing water patterns (e.g., droughts, floods), ice cap melting, heat waves, and the like. Sequestration of CO₂ accordingly can prevent or reduce it from escaping into the atmosphere.

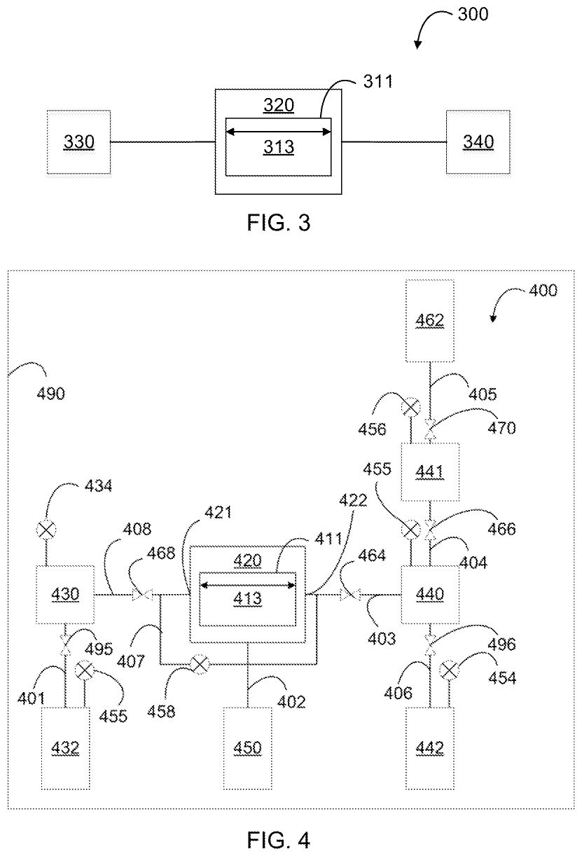

Referring now to FIG. 3, illustrated is a generalized example testing system according to one or more aspects of the present disclosure to determine permeability as a function of hydraulic gradient to evaluate caprock integrity based on non-Darcy's law flow parameters. A detailed example testing system according to one or more aspects of the present disclosure to determine permeability as a function of hydraulic gradient to evaluate caprock integrity based on non-Darcy's law flow parameters is discussed below with reference to FIG. 4.

Referring to FIG. 3, the generalized example testing system 300 takes measurements during a single test run as fluid is flowed through a caprock core sample 311 having a spatial longitudinal direction flow length 313. The system 300 includes a core container 320 for holding the caprock core sample 311, an upstream reservoir 330, and a downstream reservoir 340, each in fluid connection with the caprock core sample 311 (see FIG. 4).

The system 300 (and the system 400 of FIG. 4 discussed below) utilizes a method based on initially reaching an equilibrium such that the caprock core sample 311 is saturated with an aqueous-based testing fluid. The aqueous-based testing fluid (“testing fluid”) may be fresh water, deionized water, or a brine, for example. The equilibrium in the caprock core system 311 is such that the water pressure in the upstream reservoir 330 is identical to the water pressure in the downstream reservoir 340 and in core sample 311. Thereafter, the fluid connection between the upstream reservoir 330 and the caprock core sample 311 is closed and the water pressure in the upstream reservoir 330 is increased to a desirable value and maintained at that desirable value for the rest of the time period of the measurement. Then, the fluid connection between the upstream reservoir 330 and the caprock core sample 311 is reopened and the testing fluid at the increased pressure flows from the upstream reservoir 330, through the caprock core sample 311, and to the downstream reservoir 340. Accordingly, the pressure in the downstream reservoir 340 increases as time elapses (as the test fluid flows thereto). The pressure value in the downstream reservoir 340 is measured to determine the effective permeability of the caprock as a function of hydraulic gradient, thereby assessing the integrity of the caprock core sample 311. The following Equations are used to perform this determination:

Mass balance of testing fluid through the caprock core sample is determined by Equation 1, where t is time, x is the spatial coordinate along the longitudinal flow length of the caprock core sample, the storage factor A is determined by Equation 2, the parameter q is water mass flux and is determined by Equations 4 and 7, and the parameter p is pressure:

$$\frac{\partial q}{\partial x} = - A \frac{\partial p}{\partial t} \qquad Equation 1$$

The parameter A is determined by Equation 2, where ρ is water density, Ø is porosity, and c_{w} is water compressibility as determined by Equation 3:

$$A = ∅ \frac{d ρ}{d p} = {c}_{w} ∅ ρ \qquad Equation 2$$

The parameter c_{w} is determined by Equation 3:

$${c}_{w} = \frac{1}{ρ} \frac{d ρ}{dp} \qquad Equation 3$$

Because the testing fluid is an aqueous fluid and water density does not change significantly in the testing methods and systems of the present disclosure, the parameter A can be treated as a constant.

The water mass flux (q) is determined according to Equation 4, where k is water permeability, μ is viscosity, and K is hydraulic conductivity:

$$q = - \frac{k ρ}{μ} \frac{\partial p}{\partial x} = - K \frac{\partial p}{\partial x} \qquad Equation 4$$

The pressure difference (Δp) between the upstream reservoir and the downstream reservoir, where pᵤ is the water pressure of the upstream reservoir, and p_{d} is the water pressure of the downstream reservoir:

$$Δ p = {p}_{u} - {p}_{d} \qquad Equation 5$$

The pressure (p) along the longitudinal flow length of the caprock core sample is determined by Equation 6, where L is the flow length of the caprock core sample:

$$p = {p}_{u} - \frac{Δ p}{L} x \qquad Equation 6$$

Incorporating Equation 6 into Equation 1, the water mass flux (q) is updated, where q₀ is the water mass flux at the inlet of the caprock core sample (x=0):

$$q = {q}_{0} + \frac{A {x}^{2}}{2 L} \frac{d ( Δ p )}{d t} \qquad Equation 7$$

Combining Equation 4 and Equation 7 yields Equation 8:

$$- K \frac{\partial p}{\partial x} = {q}_{0} + \frac{A {x}^{2}}{2 L} \frac{d ( Δ p )}{dt} \qquad Equation 8$$

Integrating Equation 8 where x=0 to x=L, where p=pᵤ at x=0, and where p=p_{d} at x=L, the hydraulic conductivity K can be updated as determined by Equation 9:

$$K = \frac{{q}_{0} L}{Δ p} + \frac{A {L}^{2}}{6} \frac{d ( ln Δ p )}{d t} \qquad Equation 9$$

Finally, Equation 9 allows the determination of hydraulic gradient under transient flow conditions (i), where g is gravitational acceleration and Δz is the elevation difference between the inlet to the caprock core sample (from the upstream reservoir) and the outlet from the caprock core sample (to the downstream reservoir):

$$i = \frac{1}{L} ( \frac{Δ p}{ρ g} + Δ z ) \qquad Equation 10$$

Accordingly, for a given time period, the hydraulic conductivity according to Equation 9 and the hydraulic gradient under transient conditions according to Equation 10 with the measured mass flux flow rate (q₀) and the pressure difference (Δp). As such, the relationship between hydraulic conductivity and hydraulic gradient can be determined at various different time periods.

Referring now to FIG. 4, illustrated is a detailed schematic drawing of a laboratory system 400 that may be utilized to perform the methods of the present disclosure to determine caprock integrity.

Measurements can be taken during a single test run as a testing fluid is flowed through a caprock core sample 411. The system 400 includes a core container 420 for holding the caprock core sample 411, an upstream reservoir 430, one or more downstream liquid (e.g., aqueous-based fluid handling) reservoirs 440 (one shown), and one or more downstream gas reservoirs 441 (one shown).

The upstream reservoir 430 may be fluidly connected to an inlet 421 of the core container 420, which is fluidly connected to an inlet side of the caprock core sample 411. Likewise, the downstream reservoir 440 may be fluidly connected to an outlet 422 of the core container 420, which is fluidly connected to an outlet side of the caprock core sample 411.

The distance between the inlet 421 to the core container 420 and the caprock core sample 411 may vary without affecting measurements and calculations according to the embodiments of the present disclosure, as the flow path therebetween (e.g., a connecting tubing) has negligible flow resistance compared with flow in the caprock core sample 411. Thus, measurements and calculations taken in accordance with methods disclosed herein utilizing the system 400 may interchangeably refer to fluid pressure measurements at the core container 420 inlet 421 and at the inlet side of the caprock core sample 411. Similarly, because any flow path between the core container 420 outlet 422 and the outlet side of the caprock core sample 411 would have negligible flow resistance compared with flow in the caprock core sample 411, measurements and calculations taken in accordance with methods disclosed herein may interchangeably refer to pressure measurements at the core container 420 outlet 422 and at the outlet side of the caprock core sample 411. Note that in FIG. 4, the tubing 408 and 403 are directly connected to core sample 411.

The upstream reservoir 430 may be filled with an aqueous-based testing fluid. An upstream pump 432 may be fluidly connected to the upstream reservoir 430 via flow line 401 to pump the testing fluid from the upstream reservoir 430 into the caprock core sample 411 within the core container 420 via flow line 408. A downstream liquid pump 442 may be fluidly connected to the downstream liquid reservoir 440 via flow line 406 to pump testing fluid between the caprock core sample 411 within the core container 420 and the downstream liquid reservoir 440 via flow line 403.

The upstream pump 432 and downstream liquid pump 442 may be, for example, a hydraulic pump or other pump having high accuracy and high resolution, and may include precise pressure and flowrate control and measurement. The upstream pump 432 and the downstream liquid pump 442 may together be used to control fluid flow through the caprock core sample 411 within the core container 420, including the pressure and flow rate of the fluid through the caprock core sample 411. For example, the system 400 may include flow meters (not shown), which may be positioned upstream of the inlet 421 and downstream of the outlet 422, respectively, to measure the mass flow rates at the inlet 421 and outlet 422 of the caprock core sample 411 within the core container 420. In some embodiments, the upstream pump 432 and/or the downstream liquid pump 442 may be provided with a flow meter to measure the flow rates from the respective pumps (not shown).

One or more downstream gas reservoirs 441 (one shown) may be fluidly connected to downstream liquid reservoir 440. A downstream gas pump 462 may be fluidly connected to the downstream gas reservoir 441 via flow line 405 to pump the gas fluid into gas reservoir 441. As shown, the downstream gas reservoir 441 may be fluidly connected to downstream liquid reservoir 440 via flow line 404.

The purpose of the downstream gas pump 462 and downstream gas reservoir 441 is to pump gas (e.g., air) into the system 400 downstream. Gas (air) has a larger compressibility compared to aqueous-based fluid (e.g., the testing fluid collected in the downstream liquid reservoirs 440). As a result, the downstream gas pump 462 and the one or more downstream gas reservoirs 441 (one shown) can be used (via gas) to slow down the pressure rise at the outlet 422 of the caprock core sample 411 within the core container 420. Therefore, advantageously, the value of the second term of Equation 9 above

$$( \frac{A {L}^{2}}{6} \frac{d ( ln Δ p )}{dt} )$$

is reduced and measurement accuracy is increased because if pressure increases too quickly, the determination of the derivative with respect to time (the second term of Equation 9) involves a relatively large degree of uncertainty.

The downstream gas pump 462 may be, for example, a hydraulic pump or other pump having high accuracy and high resolution, and may include precise pressure and flowrate control and measurement. The downstream gas pump 462 may be used to provide the initial gas pressure in gas pressure reservoir 441. Before the fluid flow occurs from the upstream reservoir 430 to core sample 411, the valve 470 is turned off at the fluid line 405.

The flow length 413 of the caprock core sample 411 is measured along the dimension of the caprock core sample 411 parallel to the direction of the flow of testing fluid through the caprock core sample 411 during testing. For example, when testing fluid is flowed through an axial length of a caprock core sample 411 during testing, the flow length 413 of the caprock core sample 411 is the axial (longitudinal) length of the caprock core sample 411. As shown in FIG. 4, the caprock core sample 411 may be aligned axially along the axial length of the caprock core sample 411 between the inlet 421 and outlet 422, such that during testing, testing fluid may flow from the inlet 421, through the axial length dimension of the caprock core sample 411 (the flow length 413), and out the outlet 422.

A confining pump 450 may be connected to the core container 420 to apply a confining pressure around the caprock core sample 411. For example, the caprock core sample 411 may be placed in a sample cell, which may surround the caprock core sample 411 by an enclosed sleeve having an inlet side in fluid communication with the inlet 421 and an outlet side in fluid communication with the outlet 422. The enclosed caprock core sample 411 may be positioned within the core container 420, such that fluid may be flowed from the upstream reservoir 430 through the enclosed caprock core sample 211. The confining pump 450 may pump a confining fluid (e.g., an aqueous-based fluid, an oil-based fluid, or gas) into the core container 420 around the enclosed caprock core sample 411.

A plurality of pressure sensors may be used to monitor the pressure conditions at different locations in the system during testing of the caprock core sample 411. Pressure sensors may include high accuracy pressure transducers, including for example, piezoelectric pressure sensors, strain gauge pressure transducers, capacitance pressure transducers, potentiometric pressure transducers, and the like.

For example, and as shown in FIG. 4, a pressure sensor 455 may be in communication with the upstream pump 432; a pressure sensor 434 may be in communication with the upstream reservoir 430; a differential pressure sensor 458 may be in communication with flow line 407; a pressure sensor 454 may be in communication with the downstream liquid pump 442; a pressure sensor 455 may be in communication with downstream liquid reservoir 440; and a pressure sensor 456 may be in communication with downstream gas reservoir 441. It is to be appreciated that other pressure sensors may be used to determine various pump pressures or various in-line flow line pressures throughout the system 400, depending on the particular configuration of the system, without departing from the scope of the present disclosure.

The system 400 may comprise various valves throughout the system 400 for controlling flow through the various flow lines. For example, as shown in FIG. 4, the system 400 may include a valve 495 between the upstream pump 432 and the upstream reservoir 430 along flow line 401, a valve 468 between the upstream reservoir 430 and the core container 420 along flow line 408, a valve 464 between the core container 420 and the downstream liquid reservoir 440 along flow line 201, a valve 496 between the downstream reservoir 440 and the downstream pump 442 along flow line 406, a valve 466 between the downstream liquid reservoir 440 and the downstream gas reservoir 441 along flow line 404, and a valve 470 between downstream gas pump 462 and downstream gas reservoir 441. Each of valves 495, 468, 464, 496, 466, and 470 may be opened or closed to allow or prevent flow, respectively, through the caprock core sample 411 within the core container 420. It is to be noted that either one or both of valves 464 and 466 may be included, without departing from the scope of the present disclosure. Further, it is to be appreciated that a valve may be located between the confining pump 450 and the caprock core container 420 along flow line 402, a valve may be located along flow line 407, and a valve may be located between downstream gas reservoir 441 and downstream gas pump 462 along flow line 405 (e.g., valve 470), without departing from the scope of the present disclosure.

Optionally, the system 400 may be placed in an oven 490 with a constant temperature such that measurement tests may be conducted under an isothermal condition. In some embodiments, the oven 490 may be set to a temperature corresponding with a downhole temperature of a formation of interest, from which the caprock core sample 411 was taken.

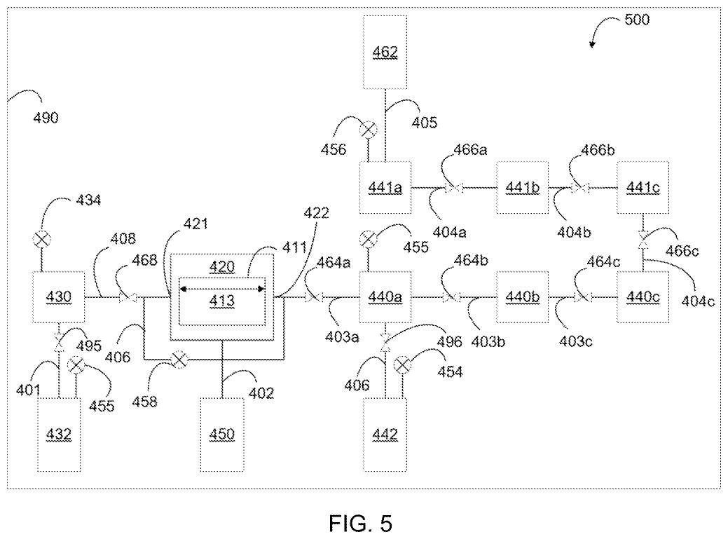

Referring now to FIG. 5, with continued reference to FIG. 4, where like reference numerals refer to like structures, including those with designated with lower case “a,” “b,” and “c.” FIG. 5 provides an alternative testing system 500, wherein a series of downstream liquid reservoirs 440a-c and a series of downstream gas reservoirs 441a-c are provided. For example, a variety of volumes can be selected for the downstream gas reservoirs for testing. It is to be appreciated that although three downstream liquid reservoirs 440a-c are shown in FIG. 5, one downstream liquid reservoir, two downstream liquid reservoirs (in series), or greater than three downstream liquid reservoirs (in series) may be included in the system 500. Similarly, it is to be appreciated that although three downstream gas reservoirs 441a-c are shown in FIG. 5, one downstream gas reservoir, two downstream gas reservoirs (in series), or greater than three downstream gas reservoirs (in series) may be included in the system 500.

As shown, the caprock sample 411 may be fluidly connected to the downstream liquid reservoir 440a along flow line 403a, the downstream liquid reservoir 440a may be fluidly connected to downstream liquid reservoir 440b along flow line 403b, and the downstream liquid reservoir 440b may be fluidly connected to downstream reservoir 440c along flow line 403c, each which may collect testing fluid flowing from the caprock core sample 440a.

With continued reference to FIG. 5, downstream gas pump 462 may be fluidly connected to the downstream gas reservoir 441a along flow line 405 to pump the gas fluid into the downstream reservoir system, including downstream gas reservoirs 441a-c and downstream liquid reservoirs 440a-c. As shown, the downstream gas reservoir 441a may be fluidly connected to downstream gas reservoir 441b along flow line 404a, the downstream gas reservoir 441b may be fluidly connected to downstream gas reservoir 441c along flow line 404c, and downstream gas reservoir 441c may be fluidly connected to downstream liquid reservoir 440c along flow line 404c.

Various valves are provided between the in-series downstream liquid reservoirs 440a-c and the downstream gas reservoirs 441a-c. As shown in FIG. 5, valve 464a may be located between core container 420 and downstream liquid reservoir 434a along flow line 403a, 464b may be located along flow line 403b, valve 464c may be located along flow line 403c, valve 466c may be located along flow line 403c, valve 466b may be located along flow line 404b, and valve 466a may be located along flow line 403a. It is to be appreciated that other valves may be used to control fluid and gas flow throughout the system 500, depending on the particular configuration of the system, without departing from the scope of the present disclosure. Moreover, it is to be appreciated that a lesser number (including one) of valves may be provided between the plurality of downstream liquid reservoirs 440a-c and a lesser number (including one) of valves may be provided between the plurality of downstream gas reservoirs 441a-c.

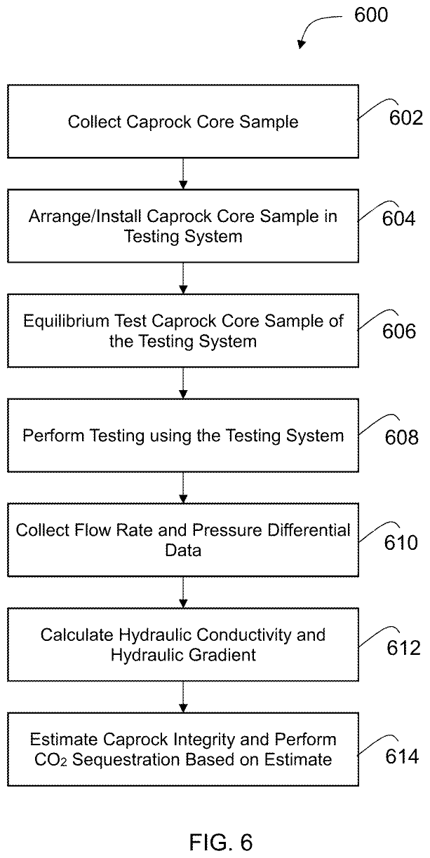

Referring now to FIG. 6, illustrated is a flowchart of an exemplary method 600 for use with the testing apparatus in one or more aspects of the present disclosure to determine hydraulic conductivity as a function of hydraulic gradient for evaluating caprock integrity. FIG. 6 will be described with reference to FIG. 4.

As shown, in step 602, a caprock core sample 411 is collected. The caprock core sample 411 may be collected using a coring process in a vertical direction through a caprock, such as introducing a coring tool into a caprock to drill and collect the caprock core sample(s) 411. The caprock core sample 411 collected at step 602 may be collected from a caprock above a saline aquifer or a depleted oil and gas well intended for evaluation or use as a CO₂ sequestration and storage reservoir. The caprock core sample 411 may be substantially cylindrical in shape and have a diameter in the range of about 1 inch (2.54 cm) to about 4 inches (10.16 cm), encompassing any value and subset therebetween, such as about 1 inch to about 2 inches, or about 2 inches to about 4 inches, or about 1.3 inches to about 1.7 inches, or about 2.3 inches to about 3.7 inches. In one or more aspects, the caprock core sample 411 may have an axial length, the axial length in the range of 1 inch (2.54 cm) to about 2 inches (5.08 cm), encompassing any value and subset therebetween, such as about 1 inch to about 1.5 inches, or about 1.5 inches to about 2 inches, or about 1.3 inches to about 1.7 inches. Other shapes and sizes may also be used without departing from the scope of the present disclosure, provided that they can be installed in the core container 420 (e.g., the configuration and size of the core container 420 as part of the testing system 400). However, the caprock core sample 411 must have an axial length in a vertical direction because the imbibition of gases or other fluids through the caprock is along the vertical direction. In one or more aspects, the caprock core sample 411 may have a diameter of about 1 inch and an axial length of about 1 inch.

Step 604 involves arranging (installing) the caprock core sample 411 in the testing system 400 within the core container 420. The core container 420 is installed between the upstream reservoir 420 and the downstream liquid reservoir 440 via inlet 412 and outlet 422, respectively. During installation, at least the valves 468 and 464 are closed.

The core container 420 may withstand a radial confining pressure in the range of 500 psi to 10,000 psi, encompassing any value and subset therebetween, such as in the range of about 500 psi to about 1500 psi, or about 500 psi to about 2,500 psi, or about 2,500 psi to about 5,000 psi, or about 1,000 psi to about 2,500 psi, or about 5,000 psi to about 10,000 psi, or about 7,500 psi to about 10,000 psi. As described above, the caprock core sample 411 may be enclosed in a sleeve within the core container 420, which may include multiple sleeve layers, such that fluid flowing through the caprock core sample 411 and the provided confining fluid (within the sleeves) are separated. The sleeves may be separate or integrated with the core container 420, without departing from the scope of the present disclosure. A confining fluid may be introduced into the core container 420, thus not contacting the caprock core sample 411. In one or more aspects, the confining fluid may be injected into a bottom side of the core container 420 to fill the core container with confining fluid using a confining pump 450 through flow line 402, while incoming confining fluid may expel air through a top side of the core container 420 (not shown).

The confining fluid and the sleeved caprock core sample 411 in the filled core container 420 may be locked into the core container 420 after no air is present in the core container 420. A confining pressure may then be applied to the caprock core sample 411 using the confining pump 450, such as by pumping more confining fluid to the space between the sleeved caprock core sample 411 and the core container 420 (e.g., within the sleeve). For example, after the core container 410 is filled with confining fluid and no air is present, the core container 420 is locked in, a confining pressure may be applied around (radially) the sleeved caprock core sample 411. The confining pressure may be in the range of about 400 psi to about 700 psi, encompassing any value and subset therebetween, such as in the range of about 400 psi to about 500 psi, or about 500 psi to about 600 psi, or about 450 psi to about 550 psi, or about 500 psi to about 700 psi, or about 600 psi to about 700 psi. In some embodiments, the confining pressure applied to the caprock core sample 411 is at least about 500 psi greater than the pore pressure in the caprock core sample 411.

With continued reference to FIG. 6, in step 606, the testing system 400 is equilibrated. More specifically, in step 606, the upstream pump 432 and the downstream liquid pump 442 at each end of the caprock core sample 411 are used to pump testing fluid (e.g., water) into the caprock core sample 411 to saturate the caprock core sample 411 to achieve water pressure equilibrium between the upstream reservoir 430 and the downstream water reservoir 440. Water may further be circulated between flow lines 403 and 408 along flow line 407 to achieve the desired equilibrium. Gas is also pumped into gas reservoir 441 at the planned equilibrium pressure using downstream gas pump 462. During the equilibrating process, valve 466 is closed. After equilibrium in Step 606, valve 470 in fluid line 405 is closed and valve 466 is opened.

During Step 604, the valve 466 is closed and valves 495, 468, 496, and 464 are opened from their closed state. Accordingly, fluid communication within flow line 408 between the upstream reservoir 430 and the caprock core sample 411 is permitted by opening valves 495 and 468, fluid communication within flow line 403 between the downstream liquid reservoir 440 and the caprock core sample 411 is permitted by opening valve 464, and fluid communication within flow line 407 is permitted. The valve 470 is also opened to provide gaseous flow to the downstream gas reservoir 441 until a predetermined equilibrium gas pressure in the downstream gas reservoir 441 is reached. Upon reaching the predetermined equilibrium gas pressure the downstream valves 496 and 470 may then be closed. Upon saturation of the caprock core sample 411 with the testing fluid, valve 466 is opened (or optionally one or more of 464b,c and one or more of 466a-c of FIG. 5 are opened). Upon reaching the desired pressure equilibrium, valve 468 is closed and valve 466 is opened.

Referring now to step 608, the flow test is performed (conducted) on the caprock core sample 411. Specifically, in this step, the valve 468 is closed (from step 606), such that fluid communication between the upstream reservoir 430 and the caprock core sample 411 is not permitted. Testing fluid is injected (e.g., pulsed) from the upstream pump 432 and into the upstream reservoir 430 until the water pressure in the upstream reservoir 430 reaches a desired value larger than the equilibrium pressure mentioned in Step 606. Thereafter, the valve 468 is opened and the testing fluid is allowed to flow between the upstream reservoir 430 and the downstream liquid reservoir 440 through the caprock core sample 411. The pressure in the upstream reservoir 430 remains constant. The pressure differential between the two ends of the caprock core sample 411 is monitored and measured at this step and the flow rate of the testing fluid is measured using the upstream pump 432. If no flow rate or pressure differential data is measured, the pressure within the upstream reservoir 430 is increased at least two-fold (e.g., after about 3 hours or more) and the testing is repeated. It is noted that in non-Darcy flow, a flow rate of zero for some hydraulic gradients can be expected (see FIG. 2).

Step 610 involves collecting (compiling) water flow rate and pressure differential data (i.e., between the inlet 421 and the outlet 422 of the caprock core sample 411) that were obtained during testing at step 608. This data is collected as a function of time.

Step 612 involves calculating the hydraulic conductivity and hydraulic gradient using Equations 9 and 10 provided herein, respectively, based on the collected flow rate and pressure differential data in step 610. As such, the relationship between the hydraulic gradient and the hydraulic conductivity can be measured as a function of time.

In step 614, the caprock integrity of the formation reservoir (e.g., saline aquifer or depleted oil and gas well) is estimated from the calculations performed in step 612. Specifically, the CO₂ imbibition into caprock is calculated using the measured hydraulic conductivity as a function of hydraulic gradient. Based on the estimation determining whether to perform a CO₂ sequestration operation in the formation reservoir and performing the CO₂ sequestration operation when the caprock integrity is estimated to be sufficient.

Accordingly, the present disclosure advantageously comprises methods and systems for the determination of caprock integrity using a single, economical test based on non-Darcy parameters for hydraulic conductivity and hydraulic gradient.

Embodiments disclosed herein include:

A. A method including determining a hydraulic gradient and a hydraulic conductivity of a caprock core sample based on non-Darcy flow using a testing system comprising a core container comprising an upstream inlet, a downstream outlet, and a confining pressure pump in fluid communication with the core container, an upstream reservoir in fluid communication with the upstream inlet and comprising a first upstream valve for selectively controlling fluid flow between the upstream reservoir and the caprock core sample located within the core container, an upstream pump in fluid communication with the upstream reservoir and comprising a second upstream valve for selectively controlling fluid flow between the upstream pump and the upstream reservoir, a downstream liquid reservoir in fluid communication with the downstream outlet of the core container and comprising a first downstream valve for selectively controlling fluid flow between the downstream liquid reservoir and the caprock core sample located within the core container, a downstream liquid pump in fluid communication with the downstream liquid reservoir and comprising a second downstream valve for selectively controlling fluid flow between the downstream liquid pump and the downstream liquid reservoir, a downstream gas reservoir in fluid communication with the downstream liquid reservoir and comprising a third downstream valve for selectively controlling gaseous flow between the downstream gas reservoir and the downstream liquid reservoir, and a downstream gas pump in fluid communication with the downstream gas reservoir and comprising a fourth downstream valve for selectively for controlling gaseous flow between the downstream gas pump and the downstream gas reservoir. The determining step may include closing the first upstream valve, the second upstream valve, the first downstream valve, the second downstream valve, the third downstream valve, and the fourth downstream valve, arranging the caprock core sample in the core container, wherein the core container is pressurized using the confining pump to a predetermined confining pressure, and equilibrating the testing system by opening the first upstream valve, the second upstream valve, the first downstream valve, and the second downstream valve to saturate the caprock core sample with water, opening the fourth downstream valve, thereby providing gaseous flow to the downstream gas reservoir until a predetermined equilibrium gas pressure in the downstream gas reservoir is reached, closing the second downstream valve and the fourth downstream valve when the predetermined equilibrium gas pressure is reached, opening the third downstream valve, and thereafter, closing the first upstream valve. The determining step may further include performing a flow test by opening the second upstream valve, thereby pressurizing the upstream reservoir using the upstream pump to a predetermined pressure; then keeping constant the pressure in the upstream reservoir, opening the first upstream valve, thereby flowing water between the upstream liquid reservoir and the downstream liquid reservoir through the caprock core sample, measuring flow rate data between the upstream inlet and the downstream outlet as a function of time, and measuring pressure differential data between the upstream outlet and the downstream outlet as a function of time. The method may further include collecting the flow rate data and the pressure differential data, calculating hydraulic conductivity (K) of the caprock core sample as a function of time where

$$K = \frac{{q}_{0} L}{Δ p} + \frac{A {L}^{2}}{6} \frac{d ( ln Δ p )}{dt} ,$$

and q₀ is the flow rate data at the upstream inlet at time zero (0), L is a length of the caprock core sample, A is a storage factor, and Δp is the pressure differential data, and calculating the hydraulic gradient (i) of the caprock core sample as a function of time where

$$i = \frac{1}{L} ( \frac{Δ p}{ρ g} + Δ z ) ,$$

and ρ is porosity of the caprock core sample, g is gravitational acceleration, and Δz is an elevation difference between the upstream inlet and the downstream outlet.

B. A testing system that includes a core container that includes an upstream inlet in fluid communication with the core container, a downstream outlet in fluid communication with the core container, and a confining pressure pump in fluid communication with a of the core container. The system further include an upstream reservoir in fluid communication with the upstream inlet of the core container and comprising a first upstream valve for selectively controlling fluid flow between the upstream reservoir and the caprock core sample located within the core container, an upstream pump in fluid communication with the upstream reservoir and comprising a second upstream valve for selectively controlling fluid flow between the upstream pump and the upstream reservoir, a downstream liquid reservoir in fluid communication with the downstream outlet of the core container and comprising a first downstream valve for selectively controlling fluid flow between the downstream liquid reservoir and the caprock core sample located within the core container, a downstream liquid pump in fluid communication with the downstream liquid reservoir and comprising a second downstream valve for selectively controlling fluid flow between the downstream liquid pump and the downstream liquid reservoir, a downstream gas reservoir in fluid communication with the downstream liquid reservoir and comprising a third downstream valve for selectively controlling gaseous flow between the downstream gas reservoir and the downstream liquid reservoir, and a fourth downstream valve for selectively for controlling gaseous flow between the downstream gas pump and the downstream gas reservoir.

Each of embodiments A and B may have one or more of the following additional elements in any combination: Element 1: further comprising repeating the equilibrating and performing steps if the measured pressure differential between the upstream inlet and the downstream outlet has not changed with time. Element 2: wherein installing the core container comprises enclosing at least one sleeve about the caprock core sample. Element 3: wherein the core container is pressurized using the confining pump by pumping a confining fluid into core container outside of the at least one sleeve. Element 4: wherein the upstream pump in the testing system pumps water into the upstream reservoir and the downstream pump in the testing apparatus pumps water into the downstream liquid reservoir. Element 5: wherein the caprock core sample has a diameter in the range of 1 inch to 4 inches, and an axial length in the range of 1 inch to 2 inches. Element 6: wherein the caprock core sample has a diameter of 1 inch and an axial length of 1 inch. Element 7: wherein the predetermined confining pressure is in the range of 500 psi to 5,000 psi. Element 8: wherein the predetermined confining pressure is in the range of 500 psi to 2,500 psi. Element 9: wherein the caprock core sample is collected from above a saline aquifer, the saline aquifer for sequestration of CO₂. Element 10: wherein the caprock core sample is collected from a depleted oil and gas well, the oil and gas well for sequestration of CO₂. Element 11: further comprising estimating caprock integrity of the collected caprock core sample based on the calculated hydraulic conductivity and the calculated hydraulic gradient. Element 12: further comprising performing a CO₂ sequestration operation based on calculating the caprock integrity. Element 13: further comprising a plurality of downstream liquid reservoirs fluidly connected by a plurality of valves. Element 14: further comprising a plurality of downstream gas reservoirs fluidly connected by a plurality of valves.

Element 15: further comprising a pressure sensor provided at each of the upstream pump, the downstream liquid pump, and the confining pump. Element 16: further comprising a pressure sensor provided at each of the upstream reservoir, the downstream liquid reservoir, and the downstream gas reservoir. Element 17: further comprising a plurality of downstream liquid reservoirs fluidly connected by a plurality of valves. Element 18: further comprising a plurality of downstream gas reservoirs fluidly connected by a plurality of valves.

By way of non-limiting example, exemplary combinations applicable to A and B include: Element 1 with Element 3; and Element 11 with Element 12.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains.” “containing.” “includes.” “including,” “comprises,” and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized that these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and are not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

What is claimed is:

1. A method comprising:
determining a hydraulic gradient and a hydraulic conductivity of a caprock core sample based on non-Darcy flow using a testing system comprising:
a core container comprising an upstream inlet, a downstream outlet, and a confining pressure pump in fluid communication with the core container;
an upstream reservoir in fluid communication with the upstream inlet and comprising a first upstream valve for selectively controlling fluid flow between the upstream reservoir and the caprock core sample located within the core container;
an upstream pump in fluid communication with the upstream reservoir and comprising a second upstream valve for selectively controlling fluid flow between the upstream pump and the upstream reservoir;
a downstream liquid reservoir in fluid communication with the downstream outlet of the core container and comprising a first downstream valve for selectively controlling fluid flow between the downstream liquid reservoir and the caprock core sample located within the core container;
a downstream liquid pump in fluid communication with the downstream liquid reservoir and comprising a second downstream valve for selectively controlling fluid flow between the downstream liquid pump and the downstream liquid reservoir;
a downstream gas reservoir in fluid communication with the downstream liquid reservoir and comprising a third downstream valve for selectively controlling gaseous flow between the downstream gas reservoir and the downstream liquid reservoir; and
a downstream gas pump in fluid communication with the downstream gas reservoir and comprising a fourth downstream valve for selectively for controlling gaseous flow between the downstream gas pump and the downstream gas reservoir;
wherein the determining comprises:
closing the first upstream valve, the second upstream valve, the first downstream valve, the second downstream valve, the third downstream valve, and the fourth downstream valve;
arranging the caprock core sample in the core container, wherein the core container is pressurized using the confining pump to a predetermined confining pressure;
equilibrating the testing system by:
opening the first upstream valve, the second upstream valve, the first downstream valve, and the second downstream valve to saturate the caprock core sample with water;
opening the fourth downstream valve, thereby providing gaseous flow to the downstream gas reservoir until a predetermined equilibrium gas pressure in the downstream gas reservoir is reached;
closing the second downstream valve and the fourth downstream valve when the predetermined equilibrium gas pressure is reached;
opening the third downstream valve; and
thereafter, closing the first upstream valve;
performing a flow test by:
opening the second upstream valve, thereby pressurizing the upstream reservoir using the upstream pump to a predetermined pressure; then keeping constant the pressure in the upstream reservoir;
opening the first upstream valve, thereby flowing water between the upstream liquid reservoir and the downstream liquid reservoir through the caprock core sample;
measuring flow rate data between the upstream inlet and the downstream outlet as a function of time; and
measuring pressure differential data between the upstream outlet and the downstream outlet as a function of time;
collecting the flow rate data and the pressure differential data;
calculating hydraulic conductivity (K) of the caprock core sample as a function of time where
$$K = \frac{{q}_{0} L}{Δ p} + \frac{A {L}^{2}}{6} \frac{d ( ln Δ p )}{dt} ,$$
and q₀ is the flow rate data at the upstream inlet at time zero (0), L is a length of the caprock core sample, A is a storage factor, and Δp is the pressure differential data; and
calculating the hydraulic gradient (i) of the caprock core sample as a function of time where
$$i = \frac{1}{L} ( \frac{Δ p}{ρ g} + Δ z ) ,$$
and ρ is porosity of the caprock core sample, g is gravitational acceleration, and Δz is an elevation difference between the upstream inlet and the downstream outlet.

2. The method of claim 1, further comprising repeating the equilibrating and performing steps if the measured pressure differential between the upstream inlet and the downstream outlet has not changed with time.

3. The method of claim 1, wherein installing the core container comprises enclosing at least one sleeve about the caprock core sample.

4. The method of claim 3, wherein the core container is pressurized using the confining pump by pumping a confining fluid into core container outside of the at least one sleeve.

5. The method of claim 1, wherein the upstream pump in the testing system pumps water into the upstream reservoir and the downstream pump in the testing apparatus pumps water into the downstream liquid reservoir.

6. The method of claim 1, wherein the caprock core sample has a diameter in the range of 1 inch to 4 inches, and an axial length in the range of 1 inch to 2 inches.

7. The method of claim 1, wherein the caprock core sample has a diameter of 1 inch and an axial length of 1 inch.

8. The method of claim 1, wherein the predetermined confining pressure is in the range of 500 psi to 5,000 psi.

9. The method of claim 1, wherein the predetermined confining pressure is in the range of 500 psi to 2,500 psi.

10. The method of claim 1, wherein the caprock core sample is collected from above a saline aquifer, the saline aquifer for sequestration of CO₂.

11. The method of claim 1, wherein the caprock core sample is collected from a depleted oil and gas well, the oil and gas well for sequestration of CO₂.

12. The method of claim 1, further comprising estimating caprock integrity of the collected caprock core sample based on the calculated hydraulic conductivity and the calculated hydraulic gradient.

13. The method of claim 12, further comprising performing a CO₂ sequestration operation based on calculating the caprock integrity.

14. The method of claim 1, further comprising a plurality of downstream liquid reservoirs fluidly connected by a plurality of valves.

15. The method of claim 1, further comprising a plurality of downstream gas reservoirs fluidly connected by a plurality of valves.

16. A system, comprising:
a core container comprising:
an upstream inlet in fluid communication with the core container;
a downstream outlet in fluid communication with the core container; and
a confining pressure pump in fluid communication with a of the core container;
an upstream reservoir in fluid communication with the upstream inlet of the core container and comprising a first upstream valve for selectively controlling fluid flow between the upstream reservoir and the caprock core sample located within the core container;
an upstream pump in fluid communication with the upstream reservoir and comprising a second upstream valve for selectively controlling fluid flow between the upstream pump and the upstream reservoir;
a downstream liquid reservoir in fluid communication with the downstream outlet of the core container and comprising a first downstream valve for selectively controlling fluid flow between the downstream liquid reservoir and the caprock core sample located within the core container;
a downstream liquid pump in fluid communication with the downstream liquid reservoir and comprising a second downstream valve for selectively controlling fluid flow between the downstream liquid pump and the downstream liquid reservoir;
a downstream gas reservoir in fluid communication with the downstream liquid reservoir and comprising a third downstream valve for selectively controlling gaseous flow between the downstream gas reservoir and the downstream liquid reservoir; and
a fourth downstream valve for selectively for controlling gaseous flow between the downstream gas pump and the downstream gas reservoir.

17. The system of claim 16, further comprising a pressure sensor provided at each of the upstream pump, the downstream liquid pump, and the confining pump.

18. The system of claim 16, further comprising a pressure sensor provided at each of the upstream reservoir, the downstream liquid reservoir, and the downstream gas reservoir.

19. The system of claim 16, further comprising a plurality of downstream liquid reservoirs fluidly connected by a plurality of valves.

20. The system of claim 16, further comprising a plurality of downstream gas reservoirs fluidly connected by a plurality of valves.
